(19) United States
(12) Patent Application Publication
(10) Pub. No.: US 2026/0174616 A1
(43) Pub. Date: Jun. 25, 2026

(54) GAIT TRAJECTORY CONTROL METHOD AND GAIT TRAJECTORY SYSTEM

(71) Applicants: Nanyang Technological University, Singapore (SG); Agency for Science, Technology and Research, Singapore (SG)

(72) Inventors: Lincong Luo, Singapore (SG); Ming Jeat Foo, Singapore (SG); Manoj Ramanathan, Singapore (SG); Chye Hsia Chiam, Singapore (SG); Wei Yun Yau, Singapore (SG); Wei Tech Ang, Singapore (SG)

(21) Appl. No.: 19/127,643

(22) PCT Filed: Oct. 16, 2023
(86) PCT No.: PCT/SG2023/050696

§ 371 Date: May 6, 2025

(30) Foreign Application Priority Data
Nov. 8, 2022 (SG) 10202260005X

(51) Int. Cl. A61H 3/00; A61B 5/11; A61H 1/02

(52) U.S. Cl. CPC A61H 3/00; A61B 5/112; A61H 1/0244; A61H 1/0262; A61H 2201/1215; A61H 2201/165; A61H 2201/5007

(57) ABSTRACT

Disclosed herein is a gait trajectory system and a gait trajectory control method. The gait trajectory system, comprises: a controller configured to: for a step of a user: using a dynamic movement primitives-based trajectory generation module, determine a reference trajectory based on a set of initial joint angles, a goal offset, and a forcing term; update a vector table based on the reference trajectory, wherein the vector table includes multiple sets of joint angles corresponding to at least one leg of the user; determine from the vector table a set of closest joint angles closest to a set of current joint angles; and determine from the vector table a set of target joint angles based on the set of current joint angles and a predetermined angular distance.

### CROSS-REFERENCE TO RELATED APPLICATION

This application claims the benefit of priority to the Singapore Application No. 10202260005X filed Nov. 8, 2022, the contents of which are hereby incorporated by reference in their entirety for all purposes.

### TECHNICAL FIELD

This application relates to a gait trajectory control method and a gait trajectory system suitable for gait assistance.

### BACKGROUND

With the development of robotic technology, lower-limb exoskeletons are increasingly used to provide gait assistance to a user during gait rehabilitation and for autonomous walking. The gait trajectory control method for the exoskeleton system influences the interaction between the user and the exoskeleton, and determines the therapy recovery results as well as the gait/walking performance. Conventional lower-limb exoskeleton systems lack flexibility, e.g., they can only provide gait assistance assuming a healthy gait, and are relatively limited in the gait rehabilitation assistance provided.

### SUMMARY

In one aspect, the present application discloses a gait trajectory system. The gait trajectory system comprises a controller configured to: for a step of a user: using a dynamic movement primitives-based trajectory generation module, determine a reference trajectory based on a set of initial joint angles, a goal offset, and a forcing term; update a vector table based on the reference trajectory, wherein the vector table includes multiple sets of joint angles corresponding to at least one leg of the user; determine from the vector table a set of closest joint angles closest to a set of current joint angles; and determine from the vector table a set of target joint angles based on the set of current joint angles and a predetermined angular distance.

According to another aspect, the present application discloses a gait trajectory control method. The gait trajectory control method comprises: for a step of a user: using a dynamic movement primitives-based trajectory generation module, determining a reference trajectory based on a set of initial joint angles, a goal offset, and a forcing term; updating a vector table based on the reference trajectory, wherein the vector table includes multiple sets of joint angles corresponding to at least one leg of the user; determining from the vector table a set of closest joint angles closest to a set of current joint angles; and determining from the vector table a set of target joint angles based on the set of current joint angles and a predetermined angular distance.

### BRIEF DESCRIPTION OF THE DRAWINGS

Various embodiments of the present disclosure are described below with reference to the following drawings:

FIG. 1 is a schematic diagram illustrating a gait trajectory system according to embodiments of the present disclosure;

FIG. 2 is a flowchart of a gait trajectory method according to an embodiment;

FIG. 3 is a gait trajectory plot of a vector table comprising knee joint angles and hip joint angles at iteration K of a step according to an embodiment;

FIG. 4 is the vector table of FIG. 3 at iteration K of the step;

FIG. 5 is the gait trajectory plot of FIG. 3 at iteration K+1 of the step;

FIG. 6 is the vector table of FIG. 3 at iteration K+1 of the step;

FIG. 7 is the gait trajectory plot of FIG. 3 at the last iteration of the step;

FIG. 8 is the vector table of FIG. 3 at the last iteration of the step;

FIG. 9 is a schematic of a lower limb of a user taking a step;

FIG. 10 is a schematic diagram of a controller according to an embodiment;

FIG. 11 is an example of a lower limb exoskeleton;

FIG. 12 a plot of joint angles illustrating gait trajectory learning and imitating results using DMP with different number of kernels;

FIGS. 13(a) to 13(f) are plots of joint angles of a swing leg generated using DMP with different parameters;

FIG. 14 is a plot showing the mean and standard deviation of a step length with different hip angle scaling values;

FIGS. 15(a) to 15(c) are plots showing respective swing phase gait trajectory under different control conditions;

FIG. 16 illustrates a comparison of the trajectory RMS errors in subjects with different exoskeleton control conditions;

FIGS. 17(a) to 17(c) illustrate hip trajectories of swing and stance legs for (a) reference trajectories; (b) actual trajectories in 2D path-based control; and (c) actual trajectories in 4D path-based control; and

FIG. 18 illustrates phase difference of crossing zero points between swing and stance hip joints.

### DETAILED DESCRIPTION

The following detailed description is made with reference to the accompanying drawings, showing details and embodiments of the present disclosure for the purposes of illustration. Features that are described in the context of an embodiment may correspondingly be applicable to the same or similar features in the other embodiments, even if not explicitly described in these other embodiments. Additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance as generally understood in the relevant technical field, e.g., within 10% of the specified value.

As used herein, the term “and/or” includes any and all combinations of one or more of the associated listed items.

For the sake of brevity, the term “joint angle” may be used interchangeably with any one or more of the terms “joint position”, “joint displacement”, “knee angle”, “hip angle”, “knee joint angle”, “hip joint angle”, etc., as will be understood from the context. The term “gait” may generally be used in the context of a manner of a user taking one or more steps in the process of walking, running, pacing, etc.

To aid understanding and not to be limiting, a detailed description of various embodiments of a gait trajectory system 100 and a gait trajectory control method 200 will be described below with reference to the appended figures. The gait trajectory system 100 and gait trajectory control method 200 may also be described as a system and a method that can plan or determine a gait trajectory and through controlling respective actuators, provide respective actuating forces and/or supporting forces to assist a user during gait rehabilitation or walking. For the sake of brevity, the following will describe various examples with respect to the gait trajectory of one step of a single leg of the user, but it will be understood that the system 100/method 200 may be used for multiple sequential or non-sequential steps taken by the user using one or both legs.

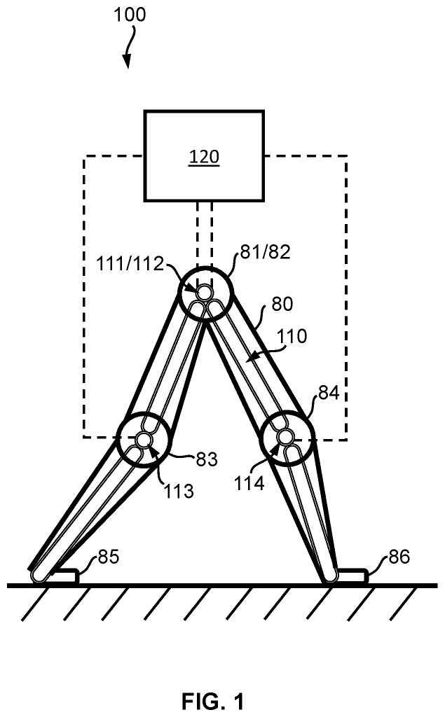

FIG. 1 is a schematic diagram of a gait trajectory system 100 according to embodiments of the present disclosure. The gait trajectory system 100 may include a lower limb exoskeleton 110 which includes a plurality of joint actuators 111/112/113/114. The lower limb exoskeleton 110 may be wearable by a user 80. In some embodiments, the joint actuators 111/112/113/114 may include hip joint actuators 111/112 and knee joint actuators 113/114. The joint actuators 111/112/113/114 may be electrical-based actuators, pneumatic-based actuators, cable/tension-based actuators, hydraulic-based actuators, magnetic-based actuators, etc.

In some embodiments, sensors may be disposed on or coupled to the exoskeleton 110 to measure a position and/or orientation of one or both lower limbs of the user 80. For example, joint angle sensors may be provided to sense or measure respective angles of the hip joints 81/82 of the user 80, or the hip joint angles of the user 80. Similarly, sensors may be provided to sense or measure respective angles of the knee joints 83/84 of the user 80 or the knee joint angles of the user 80. In some embodiments, the sensors may be integrated with the join actuators 111/112/113/114, such that the joint actuators 111/112/113/114 may perform both sensing and actuation of the respective joint angles. In some embodiments, the sensors may also be disposed or positioned adjacent to or neighbouring the respective foot 85/86 of the user 80 to measure respective position(s) and/or orientation(s) of the foot 85/86. Further, the sensors may also be configured to measure forces applied to/by the foot 85/86 to determine a contact force between the foot 85/86 and the ground or support surface.

The gait trajectory system 100 may include a controller 120 in signal communication with the plurality of joint actuators 81/82/83/84. The controller 120 may also be in signal communication with the joint angle sensors. The controller 120 may be configured to determine a set of current joint angles of the user 80, and determine a gait trajectory based on the set of current joint angles. In some examples, the set of joint angles may include a hip angle and a knee angle of one leg of the user 80. In some examples, the set of joint angles may include respective hip angles and knee angles of both legs of the user 80. In some embodiments, the controller 120 may be configured to drive the joint actuators 81/82/83/84 towards a set of target joint angles. In some embodiments, the controller 120 drives the joint actuators 81/82/83/84 based on input from the joint sensors. In some embodiments, the controller 120 may be configured to execute a gait trajectory control method.

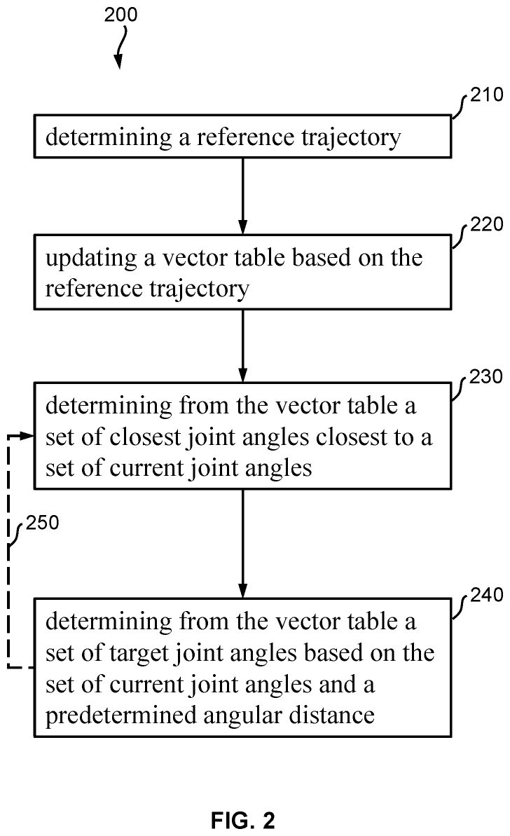

FIG. 2 illustrates a gait trajectory control method 200 according to embodiments of the disclosure. In some embodiments, the gait trajectory control method 200 may be time-invariant. In some embodiments, the gait trajectory control method 200 may be a method based on dynamic movement primitives (DMP-based method) which is suitable for modelling, planning and control of human-motor behaviours. In some embodiments, the gait trajectory control method 200 may be a path-based control method. The DMP-based method 200 is flexible for imitating or reshaping a target gait trajectory through varying a few parameters and a plurality of Gaussian kernels. Based on training data of healthy human subjects, the Gaussian kernels may be updated or learned using a machine learning method such as an explicit statistical method. Further, the method 200 may support online adjustment of the gait trajectory by exerting a forcing term on the gait trajectory when subjected to a dynamic change from the external environment.

As an exemplary embodiment, the gait trajectory control method 200 for a step of a user is disclosed. The gait trajectory control method 200 includes, in a stage 210, determining a reference trajectory based on a set of initial joint angles, a goal offset, and a forcing term. In some embodiments, the set of initial joint angles may correspond to a set of current joint angles of the user 80 at the beginning of the step. In some embodiments, the joint angles may correspond to the hip angles and/or knee angles of a user or subject undergoing gait training or gait assistance. Alternatively, in some embodiments, the joint angles may correspond to the hip angles and/or knee angles of the exoskeleton worn by the user. In some embodiments, the goal offset may correspond to a difference between an ending point of the reference trajectory and a baseline trajectory. The baseline trajectory may be a trajectory obtained from a prior healthy subject, including subjects of similar body dimensions. The baseline trajectory may be obtained from previous gait trajectory data. In some embodiments, the goal offset may correspond to a step length. For example, the goal offset may correspond to a step length of the user 80 or a step length of a prior gait trajectory. Therefore, the method 200 may further include determining the goal offset based on a step length of the user. In some embodiments, the goal offset and/or the step length may correspond to a difference between a hip joint angle of the user 80 and a hip joint angle from a database. For example, the hip joint angle from the database may be obtained from the baseline trajectory or from previous gait trajectory data. In some embodiments, the goal offset may “correct” or “modify” the ending point of the reference trajectory in relation to the baseline trajectory, therefore modifying the reference trajectory.

The forcing term may determine the shape of the reference trajectory. In some embodiments, the forcing term includes kernel weights obtained from a pre-trained machine learning model. The pre-trained machine learning model may be a dynamic movement primitives-based machine learning model which was pre-trained with the gait trajectory from healthy subjects and/or subjects whom require gait assistance. In some embodiments, the pre-trained dynamic movement primitives-based machine learning model may also be constrained by the subject's′ (both the healthy subjects and the subjects whom require gait assistance) body dimensions. In some embodiments, the forcing term may include a scaling coefficient corresponding to an ending hip angle of a previous step. In other words, the scaling coefficient may be determined based on the hip angle of one or both legs of the user for a previous step. In other embodiments, the scaling coefficient may be a constant during trajectory generation. For example, the scaling coefficient may be in a range from 1.0 to 1.2.

The gait trajectory control method 200 may further include, in a stage 220, updating a vector table based on the reference trajectory. The vector table may include multiple sets of joint angles which correspond to at least one leg of the user. The multiple sets of joint angles may include potential target joint angles corresponding to at least one leg of the user. In some embodiments, the multiple sets of joint angles may include multiple hip angles and multiple knee angles. In some embodiments, the vector table includes multiple sets of joint angles which correspond to both legs of the user.

The gait trajectory control method 200 may further include, in a stage 230, determining from the vector table a set of closest joint angles closest to a set of current joint angles; and, in a stage 240, determining from the vector table a set of target joint angles based on the set of current joint angles and a predetermined angular distance. In some embodiments, determining the set of closest joint angles and determining the set of target joint angles may be performed iteratively 250 within a single step of the user. In other words, multiple sets of current joint angles, multiple sets of closest joint angles, and multiple sets of target joint angles will be determined in a single step of the user. In some embodiments, the gait trajectory control method 200 may further include actuating the exoskeleton 110 such that the set of current joint angles is adjusted towards the set of target joint angles. For example, by controlling and actuating one or more of the joint actuators 111/112/113/114 to adjust or change the set of current joint angles based on the set of target joint angles or towards the set of target joint angles. In some examples, the exoskeleton 110 is actuated to adjust the set of current joint angles to be as close as possible to the set of target joint angles.

In some embodiments, in determining the set of closest joint angles, the controller is configured to look up the vector table, beginning from a first index of the vector table moving down the vector table and towards an end of the vector table. The controller may then select the set of joint angles from the vector that differ from the current joint angles by a smallest difference. In other words, the sets of joint angles before the first index in the vector table are not considered for determining the set of closest joint angles. For example, only sets of joint angles after the first index in the vector table are used for determining the set of closest joint angles. In some examples, the first index at the beginning of a step may correspond to or is near a start of the vector table. In some examples, the first index at the end of a step may correspond to or is near an end of the vector table.

In some embodiments, the set of target joint angles may be determined based on the set of current joint angles and a predetermined angular distance, beginning from a second index of the vector table towards the end of the vector table. In some embodiments, the second index may be an index corresponding to the set of closest joint angles in the vector table in the present iteration. In some embodiments, the predetermined angular distance corresponds to a lookahead distance. In some embodiments, the predetermined angular distance may be a fixed angle, for example 7.5°. In some embodiments, the predetermined angular distance may be selected from a range of angles. In some embodiments, the gait trajectory control method 200 further includes updating the first index to an index of the set of closest joint angles for a next iteration. For example, for each iteration, the first index may be updated after determining the set of closest joint angles closest to the set of current joint angles. In some alternative embodiments, the first index may be updated corresponding to an index of the set of target joint angles (instead of the set of closest joint angles) for a next iteration. In other words, the first index for determining the set of closest joint angles for the next iteration begins from the set of target joint angles of the previous iteration.

In some embodiments, gait trajectory control method 200 may further include determining an end point of the step responsive to the current joint angles being within a threshold from a last set of joint angles of the vector table. The end point of the step may be determined when the current joint angles are at or within a threshold of the last set of joint angles or an end of the vector table. This corresponds to the current joint angles are close to or approaching the end of the step, and hence the end point of the step is determined. When the end point of the step is determined, the method 200 further includes proceeding to determine a next step, where a new reference trajectory is determined and the vector table is updated corresponding to the new reference trajectory.

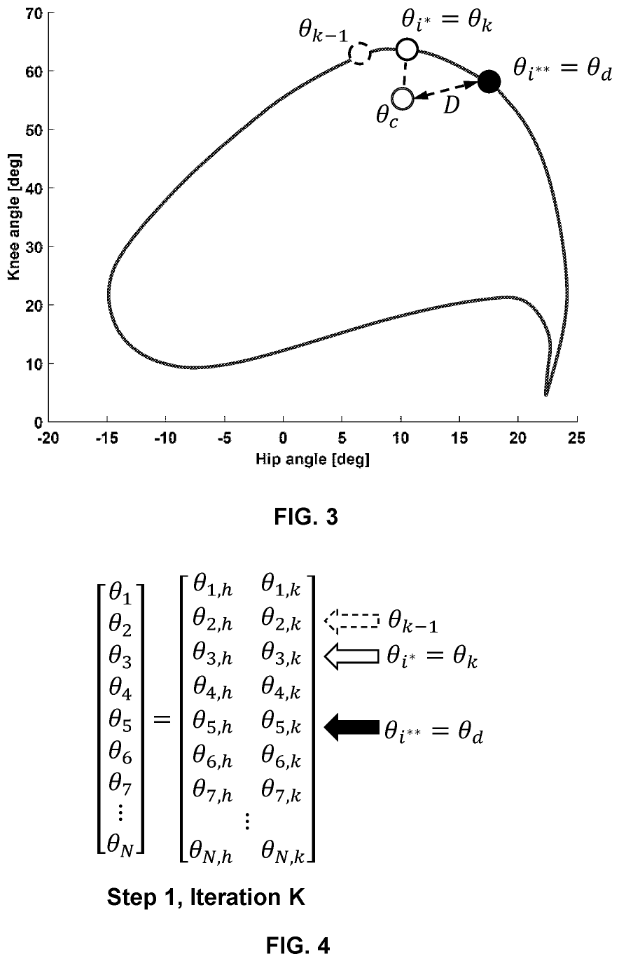

FIGS. 3 to 9 illustrate schematically various exemplary embodiments of the gait trajectory control method 200. Referring to FIGS. 3 and 4, an exemplary embodiment of a 2D plot of a vector table and the vector table at iteration k of a step is illustrated. As shown in FIG. 4, the vector table may be represented by T {i, θᵢ}, which is a vector of N set of joint angles θᵢ. Each set of joint angles θᵢ={θ_{i, h}, θ_{i, k}} includes a hip angle θ_{i, h} and a knee angle θ_{i, k}. In some examples, depending on the computational resources, the intended application, the amount of assistance required by the user 80 or other conditions, the vector may include 250 rows/sets of joint angles, 500 rows/sets of joint angles, 2000 rows/sets of joint angles, etc.

At the present iteration K, the first index for determining the set of closest joint angles corresponds to index k−1, which is the index of the set of closest joint angles of a previous iteration K−1. Beginning from the first index k−1, the method 200 determines the set of closest joint angles θᵢ* based on the set of current joint angles θ_{c}. Thereafter, beginning from the second index k, which is the index corresponding to the set of closest joint angles θᵢ* in the vector table in the present iteration, the method 200 determines the set of target join angles θᵢ** based on the set of current joint angles and a predetermined angular distance. With the target joint angles θᵢ**, the controller drives the set of current joint angles θ_{c} towards the target position θ_{d} by driving the joint actuators 111/112/113/114, optionally with positional, orientational or dynamic parameters input from the joint sensors. Further, the first index k−1 is then updated to correspond to the index k of the set of closest joint angles θᵢ* or the second index, for a next iteration.

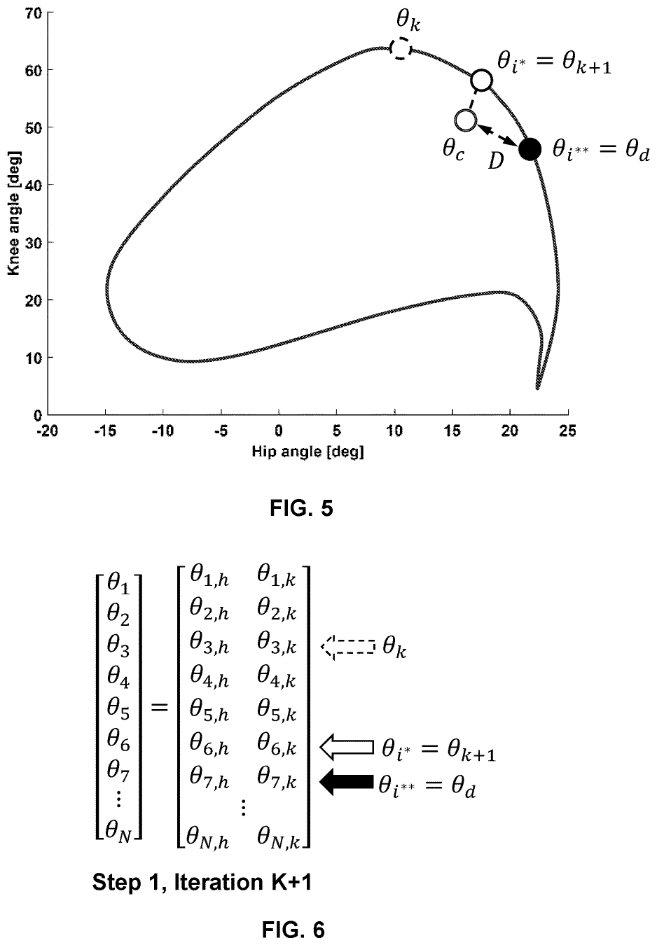

Referring now to FIGS. 5 and 6, in a next iteration K+1 of the same step, an exemplary embodiment of a 2D plot of a vector table and the vector table is illustrated. Beginning from the updated first index k, the method 200 determines a new set of closest joint angles θᵢ* based on the set of current joint angles θ_{c} which was actuated and moved by the joint actuators 111/112/113/114 in the previous iteration K. Beginning from the second index k+1, which is the index corresponding to the set of closest joint angles θᵢ* in the vector table in the present iteration K+1, the method 200 determines the set of target join angles θᵢ** based on the set of current joint angles and the predetermined angular distance. With the target joint angles θᵢ**, the controller drives the set of current joint angles θ_{c} towards the target position θ_{d} by driving the joint actuators 111/112/113/114. Further, the first index k is then updated to correspond to the index k+1 of the set of closest joint angles θᵢ* or the second index, for a next iteration K+2.

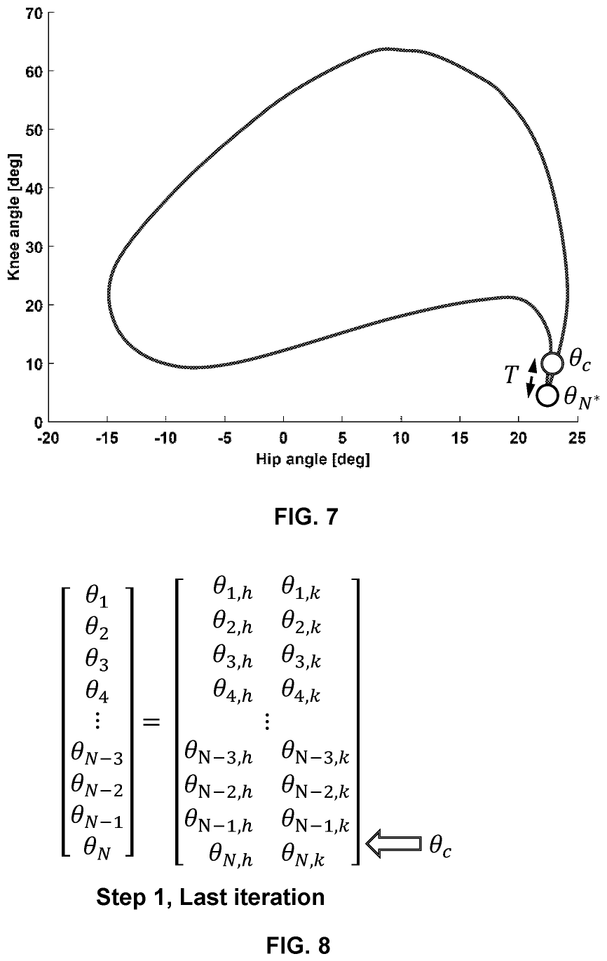

Referring now to FIGS. 7 and 8, the method 200 may include determining an end point of the current step. The end point may be determined when the set of current joint angles θ_{c} are at or within a threshold (T) from a last set of joint angles θ_{N}={θ_{N, h}, θ_{N, k}} of the vector table. In other words, when the set of current joint angles is near the end of the vector table, indicates an end point to the current step. Responsive to determining the end point of the current step, the method 200 proceeds to a next strap where a new reference trajectory is determined and the vector table is updated corresponding to the new reference trajectory.

The exemplary embodiments as illustrated in FIGS. 3 to 8 include a vector table corresponding to one leg of a subject/user, and the method may generally be referred to in this disclosure as a 2D path-based control method. It may be appreciated that vector tables which include multiple sets of joint angles which correspond to both legs of the user may also be employed in a similar manner and may generally be referred to in this disclosure as a 4D path-based control method. The 4D path-based control method may include sets of joint angles corresponding to a swing leg (active leg) and a stance leg (passive leg), as illustrated in the vector table below. In other examples, the vector table for the 4D path-based control method may correspond to a left leg and a right leg.

$$T \{ i , {θ}_{i} \} = [ \begin{matrix}{θ}_{1 , swing} & {θ}_{1 , stance} \\ {θ}_{2 , swing} & {θ}_{2 , stance} \\ {θ}_{3 , swing} & {θ}_{3 , stance} \\ {θ}_{4 , swing} & {θ}_{4 , stance} \\ {θ}_{5 , swing} & {θ}_{5 , stance} \\ {θ}_{6 , swing} & {θ}_{6 , stance} \\ {θ}_{7 , swing} & {θ}_{7 , stance} \\ ⋮ & \\ {θ}_{N , swing} & {θ}_{N , stance}\end{matrix} ] = [ \begin{matrix}{θ}_{1 , h , swing} & {θ}_{1 , k , swing} & {θ}_{1 , h , stance} & {θ}_{1 , k , stance} \\ {θ}_{2 , h , swing} & {θ}_{2 , k , swing} & {θ}_{2 , h , stance} & {θ}_{2 , k , stance} \\ {θ}_{3 , h , swing} & {θ}_{3 , k , swing} & {θ}_{3 , h , stance} & {θ}_{3 , k , stance} \\ {θ}_{4 , h , swing} & {θ}_{4 , k , swing} & {θ}_{4 , h , stance} & {θ}_{4 , k , stance} \\ {θ}_{5 , h , swing} & {θ}_{5 , k , swing} & {θ}_{5 , h , stance} & {θ}_{5 , k , stance} \\ {θ}_{6 , h , swing} & {θ}_{6 , k , swing} & {θ}_{6 , h , stance} & {θ}_{6 , k , stance} \\ {θ}_{7 , h , swing} & {θ}_{7 , k , swing} & {θ}_{7 , h , stance} & {θ}_{7 , k , stance} \\ & ⋮ & & \\ {θ}_{N , h , swing} & {θ}_{N , k , swing} & {θ}_{N , h , stance} & {θ}_{N , k , stance}\end{matrix} ]$$

wherein the current set of joint angles θ_{c} may be represented as:

$${θ}_{c} = [ {θ}_{c , h , swing} {θ}_{c , k , swing} {θ}_{c , h , stance} {θ}_{c , k , stance} ]$$

In the 4D path-based control method, the joint angles of both legs of the user 80 are taken into consideration during determination of the set of closest joint angles and during determination of the set of target joint angles for each iteration. This advantageously improves the angular synchronisation of the user during gait assistance. In some embodiments, only the swing leg is driven/actuated for the 4D-path based control method. In other words, the controller is configured to drive the joint actuators coupled to the swing leg based on the set of target joint angles, while leaving the stance leg of the user undriven or unactuated.

### DMP-Based Gait Trajectory Planning and Generation

According to various exemplary embodiments, disclosed herein is a gait trajectory system and a gait trajectory control method for the lower limb exoskeleton to provide a user with gait assistance during walking. The gait trajectory system includes a dynamic movement primitives (DMP)-based reference trajectory generation module and a path-based trajectory control module. In the trajectory generation module, DMP method is applied to learn and imitate the baseline gait trajectories collected from healthy subjects. Parameters such as the initial joint angle, scaling coefficient and goal position were used to reshape the reference trajectory. The implementation of DMP module provides a flexible manner to online adjust gait trajectory for matching different initial joint configurations in the pre-swing phase and adapting to the different step length through the settings of the corresponding parameters. In the path-based trajectory control module, the gait path is first created by using the obtained reference trajectories, followed by using a lookahead distance according to the current joint position to search for the target joint position or target joint angles in 4D joint space, in which the target joint position includes hip and knee angles of the swing and stance legs. The obtained target joint position is then used to calculate the assistive torque in the low-level controller. Unlike conventional path-based control, the path-based trajectory control as disclosed herein promotes the synchronization between the swing and stance legs as it considers the movement of both legs. The present method provides sufficient walking assistance to the user or subject with a variable gait, i.e., variation in step length and time. This allows for variance of joint angle configuration at the starting phase of each new gait/step and supports a variation in step length throughout the course of gait assistance.

Disclosed herein are three types/kinds of gait trajectory: baseline trajectory, reference trajectory and target trajectory. The baseline trajectory may be a template obtained from a gait library with data collected from healthy subjects. The reference trajectory may be the reference joint angular profile which the exoskeleton is expected to move along. The reference trajectory matches different initial joint configurations and adapt to different step length. The reference trajectory may be updated based on the baseline trajectory and the realtime joint angles/joint state at the moment of starting a new step or a new swing phase. The target trajectory may be calculated by the trajectory control module, and provides the desired control command for the controller of the assistive exoskeleton.

The baseline gait trajectory may have the advantage of ensuring the comfort of the exoskeleton control, as it is collected from healthy subjects and has human-like characteristics. In various scenarios, the baseline trajectory is not directly used as the reference trajectory without any adjustment. Such may be the case, for example, when there is a large variance in the foot touchdown location and/or when the users (especially stroke patients) encounter external disturbance(s) and should adapt their gait pattern for balance. Adjustment may be made during the change of the initial joint angle for a next swing phase or next step. To improve the stability and safety of exoskeleton control, the generated trajectory may match the joint configuration in the start of a swing phase. In addition, the reference trajectory generation may include the capability of supporting the step length adjustment to improve the mobility of exoskeletons.

The DMP approach allows easy and flexible imitation or reshaping of a target trajectory through varying/modifying a few parameters and a group of Gaussian kernels which may be obtained/learned/trained by statistical methods. Additionally, online trajectory adjustment may be enabled by exerting a forcing term in response to dynamic changes of the external environment. A DMP framework is used to model the trajectories of the hip joints and knee joints of a user. The nonlinear dynamic system of DMP may be described as:

$${τ}^{2} \ddot{y} = {α}_{y} ( {β}_{y} ( g + {g}_{offset} - y ) - τ ý ) + f \qquad ( 1 )$$

where τ is a time constant, α_{y} and β_{y} are positive constants. In some examples, τ, α_{y} and β_{y} may be respectively set as 1.0, 50.0, 12.5. y, \dot{y}, ÿ are joint angular position, velocity and acceleration, respectively. g is the goal position which y will converge to over time. f is a forcing term, which determines the shape of the trajectory. f may be expanded as follows:

$$f ( s ) = \frac{{\sum }_{i = 1}^{N} {Ψ}_{i} ( s ) {ω}_{i}}{{\sum }_{i = 1}^{N} {Ψ}_{i} ( s )} \cdot s \cdot r \qquad ( 2 )$$

$${Ψ}_{i} ( s ) = exp ( - \frac{1}{2 {σ}_{t}} {( s - {c}_{i} )}^{2} ) \qquad ( 3 )$$

where Ψᵢ are Gaussian basis function kernels, σᵢ and cᵢ are constants that determine the width and centers of the Gaussian basis function. ωᵢ are kernel weights that are learned/trained based on the kinematic data of the baseline gait trajectory. r is a scaling coefficient. The combination of g_{offset} and r is able to affect the generation of the reference trajectory. sis a phase state variable and goes from 1 to 0 for preventing or avoiding the time dependence of the nonlinear system. s may be defined as a first-order linear dynamics:

$$τ \dot{s} = - {α}_{s} s \qquad ( 4 )$$

The implementation of DMP modeling gait trajectory includes offline learning and online replaying stages. In the offline learning stage, the baseline trajectory is used as the training template to obtain the forcing term of DMP unit. The baseline gait trajectory has the advantage of ensuring the comfort of the exoskeleton control, as it is collected from healthy subjects and has human-like characteristic. The kernel weight ωᵢ may be learned by using locally weighted regression technology, as below.

$${ω}_{i} = \frac{{s}^{T} {Γ}_{i} {f}_{t}}{{s}^{T} {Γ}_{i} s} \qquad ( 5 )$$

$$s = {[ s ( 1 ) , s ( 2 ) , … , s ( p ) ]}^{T} \qquad ( 6 )$$

$${Γ}_{i} = [ \begin{matrix}{Ψ}_{i} ( 1 ) & 0 & … & 0 \\ 0 & {Ψ}_{i} ( 2 ) & … & 0 \\ ⋮ & ⋮ & ⋱ & ⋮ \\ 0 & 0 & … & {Ψ}_{i} ( p )\end{matrix} ] \qquad ( 7 )$$

$${f}_{t} = {[ {f}_{t} ( 1 ) , {f}_{t} ( 2 ) , … , {f}_{t} ( p ) ]}^{T} \qquad ( 8 )$$

$${f}_{t} = {τ}^{2} {\ddot{y}}_{t} - {α}_{y} ( {β}_{y} ( g + - {y}_{t} ) - τ {\dot{y}}_{t} ) \qquad ( 9 )$$

wherein yₜ(p), \dot{y}ₜ(p), ÿₜ(p) are the sample of joint angular position, joint angular velocity and joint angular acceleration with respective to the baseline trajectory which is collected from healthy subject walking by motion capture system. The procedure of weights learning is conducted offline, with the respective values of the kernel weights embedded for use.

A reference trajectory is generated or updated when the user is starting a new step. To generate a new reference trajectory, three extra parameters, y(0), g_{offset}, r are provided for computing iteratively during real-time control.

$$\ddot{y} ( i + 1 ) = \frac{1}{{τ}^{2}} ( {α}_{y} ( {β}_{y} ( g + {g}_{offset} - y ( i ) ) - τ \dot{y} ( i ) ) + f ( i + 1 ) ) \qquad ( 10 )$$

$$\dot{y} ( i + 1 ) = \dot{y} ( i ) + \frac{1}{τ} \ddot{y} ( i + 1 ) \qquad ( 11 )$$

$$y ( i + 1 ) = y ( i ) + \frac{1}{τ} \dot{y} ( i + 1 ) \qquad ( 12 )$$

wherein y(0) is the initial joint angle of the reference trajectory. y(0) is set as the joint angle in the starting swing phase to match the gap between the current joint position and the baseline trajectory. g_{offset} describes the difference in the ending point between the baseline trajectory and the reference trajectory, and is related to the step length, which will be presented in subsequent section. r in equation (2) may be used to scale the range of joint angles.

Subjects walking on level ground wearing exoskeletons may have a relatively large variation in foot touchdown location in the ending of the swing phase. The different foot touchdown location causes large changes of starting joint angle in the next step. Thus, if the staring point of the reference trajectory is fixed and does not match the starting joint configuration on the initial swing phase, a jerk may be produced affecting the stability of the exoskeleton control, and may even lead to falling. Therefore, the DMP-based trajectory generator can smooth the gap between the starting joint angle and the reference trajectory.

### Step Length Adjustment

In the present disclosure, while aligning the reference trajectory with the initial joint position, the DMP module provided a flexible manner to plan the reference gait trajectory allowing adjustment to the step length while maintaining the shape of the baseline trajectory. The step length describes the distance between the front and the back feet in the double phase, and is related to the hip and knee joint angles in the ending of the swing phase (the moment of heel strike).

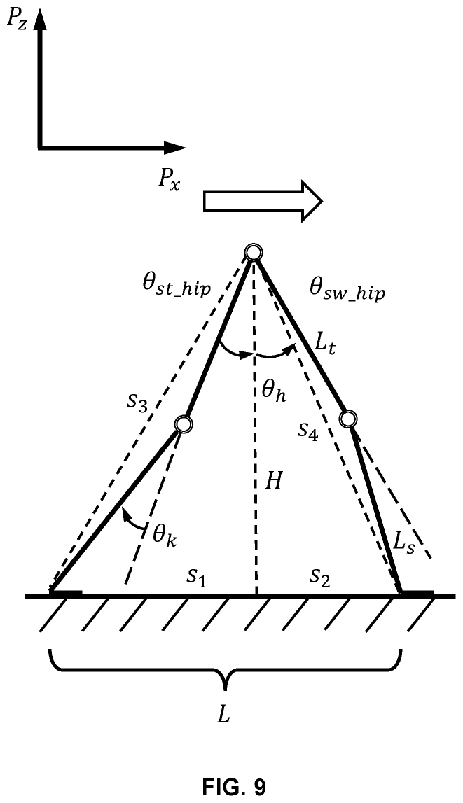

One of the common joint configuration for lower limb exoskeletons includes two active actuators on hip and knee joints, and provides leg motion in the sagittal plane. With the assumption that the foot is regarded as a point during walking, referring to FIG. 9 based on the kinematics of the exoskeleton, the step length may be expressed as follows:

$$S L = {L}_{t} \cdot ( sin ( {θ}_{s w}^{h} ) - sin ( {θ}_{st}^{h} ) ) + {L}_{s} \cdot ( sin ( {θ}_{s w}^{h} + {θ}_{s w}^{k} ) - sin ( {θ}_{st}^{h} + {θ}_{st}^{k} ) ) \qquad ( 13 )$$

wherein Lₜ, Lₛ are the length of the thigh links and shank links respectively, θ^{h}_{sw}, θ^{k}_{sw}, θ^{h}ₛₜ, θ^{k}ₛₜ are the ending hip and knee joint angle of the swing leg and the stance leg in a swing phase.

According to equation (13), the step length may be determined by the ending hip and knee angle. It is noted that the knee joint is typically kept straight or substantially straight at the moment of heel strike during walking. Therefore, it is possible to achieve a modulation in step length by adjusting the ending hip angle and fixing the ending knee angle. Referring to FIG. 9, the following kinematic constraints is as follows:

$${S}_{1} + {S}_{2} = L \qquad ( 14 )$$

$${S}_{3}^{2} - {S}_{1}^{2} = {H}^{2}$$

$${S}_{4}^{2} - {S}_{2}^{2} = {H}^{2}$$

$${S}_{1} = \frac{{L}^{2} + {S}_{3}^{2} - {S}_{4}^{2}}{2 L} \qquad ( 15 )$$

$${S}_{2} = \frac{{L}^{2} + {S}_{4}^{2} - {S}_{3}^{2}}{2 L}$$

wherein H is the vertical distance of hip joint to the ground, and L is the required step length. S₃ and S₄ are the distance between hip and ankle of the front and back legs. With the inverse kinematics, the target ending hip angle may be obtained as follows:

$${θ}_{st}^{h} = - arcsin ( \frac{{S}_{1}}{{S}_{3}} ) + arccos ( \frac{{L}_{s}^{2} - {L}_{t}^{2} - {s}_{3}^{2}}{- 2 {L}_{t} {S}_{3}} ) \qquad ( 16 )$$

$${θ}_{s w}^{h} = - arcsin ( \frac{{S}_{2}}{{S}_{4}} ) + arccos ( \frac{{L}_{s}^{2} - {L}_{t}^{2} - {s}_{4}^{2}}{- 2 {L}_{t} {S}_{4}} )$$

wherein θ^{h}ₛₜ and θ^{h}_{sw} are the ending hip angles of the stance leg and swing leg respectively. Lₛ and Lₜ are the length of shank links and thigh links respectively. With the ending angle, the goal offset θ^{h}_{offset} for hip joint trajectory DMP may be calculated and then provided to equation (10) for online generation of the reference trajectory. According to the above embodiments, adjustment to the step length may be a continuous adjustment, i.e the step length may be any possible step length value determined based on the ending hip and knee angle.

In other embodiments, the step length may be varied by a discrete value, such as a scaling variable μ. Therefore, the step length may be adjusted or varied by scaling the ending hip angle and fixing the ending knee joint angle as follows:

$${θ}_{sw , st}^{h} = μ \cdot {θ}_{g , st , sw}^{h} \qquad ( 17 )$$

$${θ}_{sw , st}^{k} = {θ}_{g , st , sw}^{k} \qquad ( 18 )$$

where μ is the scaling variable to adjust step length. θ^{h}_{g,st,sw} and θ^{k}_{g,st,sw} are the ending hip and knee angles from the baseline trajectories, respectively. With the ending angle, the goal offset θ^{h}_{offset} for the hip joint trajectory DMP may be calculated and then provided to equation (10) for the online generation of the reference trajectory.

### Path-Based Trajectory Control

With reference to FIGS. 3 and 4, the reference trajectory generated by the DMP module is employed to build a gait path consisting of hip and knee joint angles. Instead of directly configuring the assistive torque based on the path, the method searches the target point in hip-knee joint angle space based on a lookahead distance. As illustrated in FIG. 4, T {i, θᵢ} represents the vector table to store the gait path, with element θᵢ corresponding to the vector including a hip angle θₕ and a knee angle θₖ. Further, θ_{c} is the vector corresponding to the current hip angle and current knee angle.

In exemplary embodiments, the target position θ_{d} of an interation is presented as follows:
Step 1: Update the vector table T {i, θᵢ} using the reference trajectory obtained in the earlier section. The index i₀=0 is initialized for a new step.
Step 2: From the vector table, search and determine the closest point θᵢ* in the gait path which has the smallest distance to the current position. The vector of θᵢ*-θ_{c} may be regarded as the normal vector perpendicular to the gait path:

$$min : {‖ {θ}_{i *} - {θ}_{c} ‖}_{2} \qquad ( 19 )$$

wherein iₖ₋₁≤i*≤M, iₖ₋₁ is the index of the last closest point, and M is the length of vector table T {i, θᵢ}. Searching the new closest point with starting from the last closest point may reduce the computation time and avoid the issue of the index jumping backward during the search.
Step 3: From the vector table, search and determine the target point θᵢ** which has a distance D to the current position θ_{c}.

$${‖ {θ}_{i *} - {θ}_{c} ‖}_{2} \geq D \qquad ( 20 )$$

wherein i*≤i**≤M, D is a predefined distance which affects the assistance level and the move speed of target point. In this example, D is set to the value of 7.5°.
Step 4: Update the target position θ_{d} and the searching index, and move again to Step 2 for a next iteration, unless the current gait cycle ends.

$${θ}_{i * *} \to {θ}_{d} \qquad ( 21 )$$

$${i}^{*} \to {i}_{k} \qquad ( 22 )$$

The proposed path-based trajectory control method contributes several advantages to exoskeleton assistive control. Apart from providing movement assistance, the control method also guides leg motion to facilitate the inter-joint coordination between hip and knee joint. In contrast to methods which only provide robotic assistance in the normal direction, the method of the present disclosure may also produce the assistive force in the tangential direction. The assistive force along the trajectory has great significance for the subject with severe motor disorders. Moreover, the predefined lookahead distance plays an effective role of modulating the normal forces and tangential forces. When the user or subject's leg(s) are close to the gait path, more assistive force is exerted in the tangential direction. If the subject's legs deviate significantly from the reference trajectory, a larger assistive force is exerted in the normal direction to correct leg motion.

Further, in comparison to time-dependent trajectory tracking control which varies and controls the target joint angle with time, the present method enables users to have variations in step time as the target joint angle is time-independent or time-invariant rather than according to fixed time periods. Therefore, when a sudden external disturbance is present, the user can adapt the time of touchdown to maintain balance.

### Stance and Swing Legs Synchronization

The present method combines the hip and knee angles/trajectories of both the swing legs and the stance legs, creating a four-dimensional vector table for the target point searching. It was found that the present method effectively synchronizes the swing leg movements with the stance leg movements, and offers a significant improvement over conventional exoskeleton controls that either ignore the stance leg or computes the swing and stance leg trajectories separately. Improving the synchronization between the swing and stance legs while allowing for non-identical walking patterns in the two legs enables the patient to train towards a more natural and more stable walking pattern.

According the various embodiments of the present disclosure, the target point searching may be implemented in 4D joint space using the above searching approach. The method of searching with both leg joint angles is referred to herein as 4D path-based trajectory control. 4D path-based trajectory control considers the movement of both the swing and stance legs, and promotes synchronization of both legs. For ease of expression, embodiments of the method which searches for current position or target position in a single leg may be referred to as 2D path-based control. Embodiments of the present method that include searching for a current position or target position in both legs is referred to herein as 4D path-based control.

Low-Level Control with Gravity Compensation

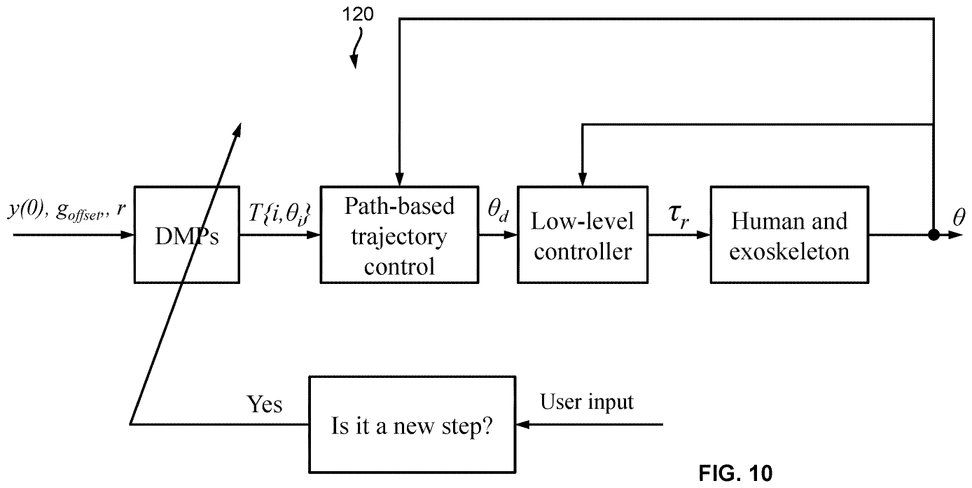

FIG. 10 is a control schematic of a controller 120 of the gait trajectory system 100 according to various embodiments. Referring to FIG. 10, the target joint angles θ_{d} are input into the low-level controller. The task of the low-level control loop of the lower limb exoskeleton is to provide sufficient assistance to guide or support the user's legs to track the desired angular position. Impedance control may be used to provide compliant interaction between human and robot. To reduce the disturbance brought by the dynamics of the exoskeleton and the user's legs, a feedforward controller with impedance control and gravity compensation is employed to correct trajectory tracking errors. With gravity compensation, compliance of the exoskeleton control is improved as gravity compensation permits the relatively low stiffness impedance control to provide sufficient assistive force. An exemplary control equation is as follows:

$${τ}_{r} = {K}_{s} \cdot ( {θ}_{d} - θ ) - {B}_{d} \cdot \dot{θ} + G ( θ ) \qquad ( 23 )$$

where τᵣ is a 2×1 vector containing the required joint torque. θ and \dot{θ} are 2×1 vectors containing the actual joint angular position and velocity of the hip and knee joints. θ_{d} is the desired joint angle vector. Kₛ and B_{d} are respectively the stiffness and damping of the impedance controller. G(θ) is the gravity compensation term.

### EXPERIMENTS AND RESULTS

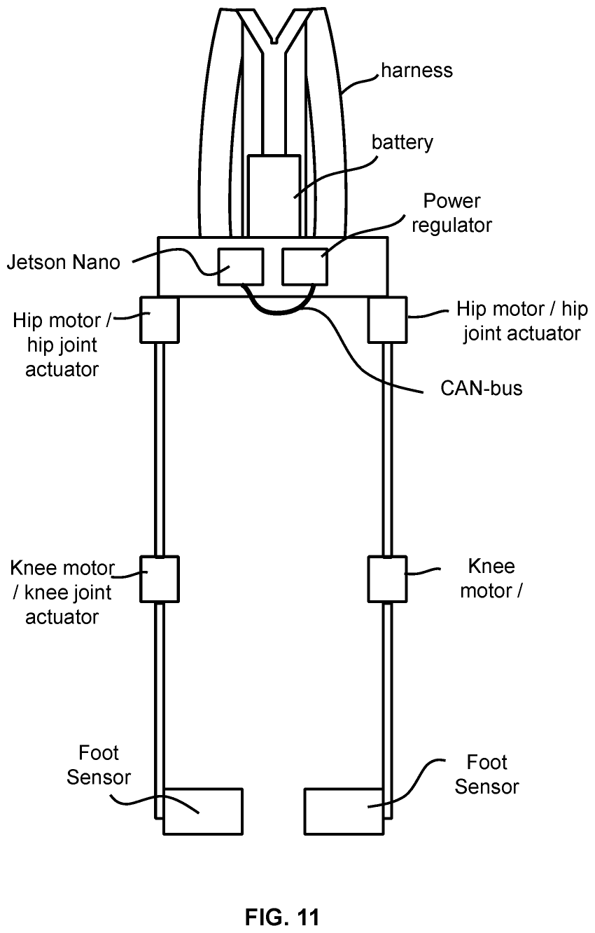

To verify the feasibility of the present method, a lower limb exoskeleton (Phoenix) as illustrated in FIG. 11 was used to implement the proposed control. The lower limb exoskeleton includes two active actuators for the hip joints, two active actuators for the knee joints, two passively adjustable ankle joints, as well as an IMU module for measuring torso inclination angle. The lower limb exoskeleton also includes a CAN-bus communication port and robot SDK to obtain exoskeleton status information and send position/torque command to its microcontroller. The developed control method runs on the NVIDIA Jetson Nano board installed with Robot Operating System (ROS). A power regulator was mounted to get the power from the equipped battery, and foot pressure sensors were applied to detect the gait events.

### Experimental Protocol

A focus of the experiment includes studying aspects of gait trajectory generation and assistive control strategies. To evaluate the method, the performance of DMP-based approach was first validated in simulation, thereafter level-ground walking experiments with subjects were conducted to demonstrate the assistive control. Five healthy subjects (two females and three males) who had never suffered from walking ability impairments were involved in the walking experiments, where they were required to walk overground with the exoskeleton in three different control conditions: unassisted control, 2D path based control and 4D path-based control. The subjects used a remote button to control the starting of the swing phase, and the ending of the swing phase was detected by kinematics data and foot pressure sensors. In the unassisted control condition, the exoskeleton was kept in zero-torque status, where the participants could move the legs by their own will as the exoskeleton joints were highly backdriven. To simulate the lower limb motor impairment, the passive torque generated by friction, inertial and gravity force was not counteracted by the exoskeleton, which would have the effect of altering the shape of subjects' gait trajectory in the swing phase from the typical healthy shape. The unassisted control condition was used as the comparison with the other two assistive control conditions. In 2D and 4D path-based control conditions, the exoskeleton provided leg movement assistance by implementing the same control strategy proposed in previous sections, but the only difference between the two conditions was the latter searched the target trajectory in the 4D join space consisting of hip and knee angle of both swing and stance legs. In each session of the above three conditions, subjects walked straight about 50m wearing the exoskeleton with crutch aid at their preferred and comfortable pace. To assess the exoskeleton assistance, the trajectory rootmean-square (RMS) errors, step length, and step time in swing phase were measured during walking experiments.

### Gait Trajectory Generation and Step Length Scaling

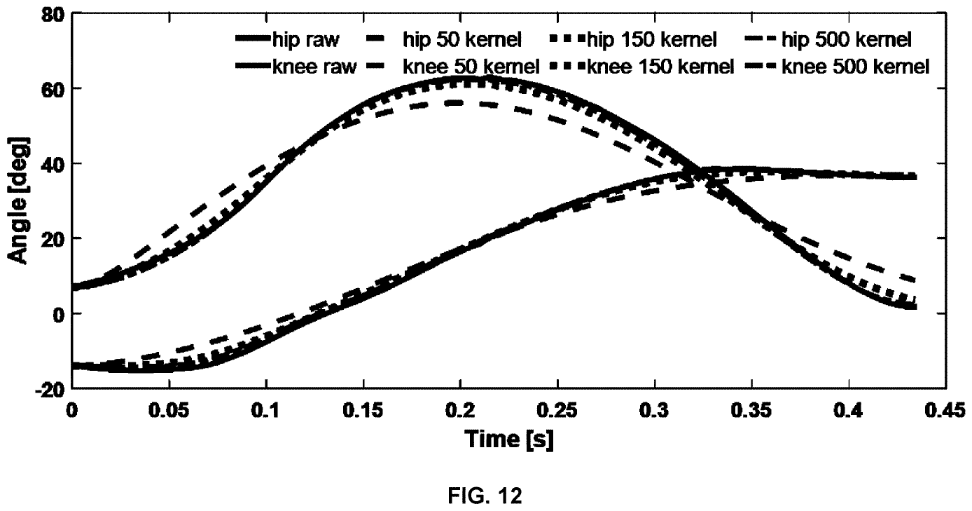

As stated previously, DMP was employed to learn and imitate the baseline trajectory, and thereafter used to generate the reference trajectory for each step of the user based on the parameter setting. To verify the capability of DMP units learning trajectory, the trajectory fitting results of swing hip and knee joints with different number of kernels are shown in FIG. 12, where the starting point of reference trajectories was set as the first point of baseline trajectories, the scaling coefficient was set at 1.0, and the goal offset was set to 0°. Among the trajectories, the raw trajectories are the baseline trajectories of hip and knee, and the others are the corresponding trajectories generated by DMP units. It may be seen from FIG. 12 that the generated trajectories are able to imitate the baseline trajectory for both hip and knee joints. Increasing the number of Gaussian basis function kernels results in generated trajectories which follow the respective baseline trajectories better. This may be associated with a higher computational cost to the controller board, nonetheless, a good balance between the fitting accuracy and computation complexity can be obtained in experiments with the number of kernels set to 150.

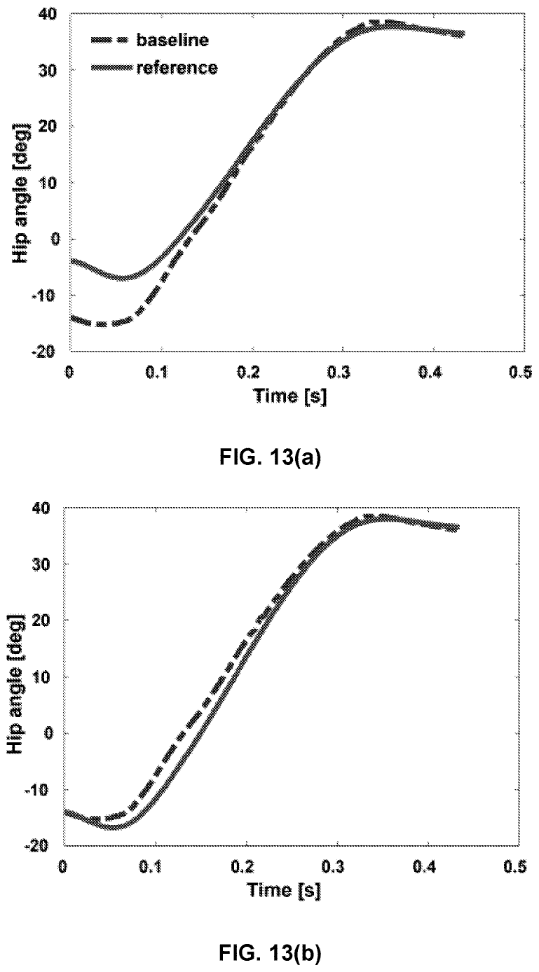

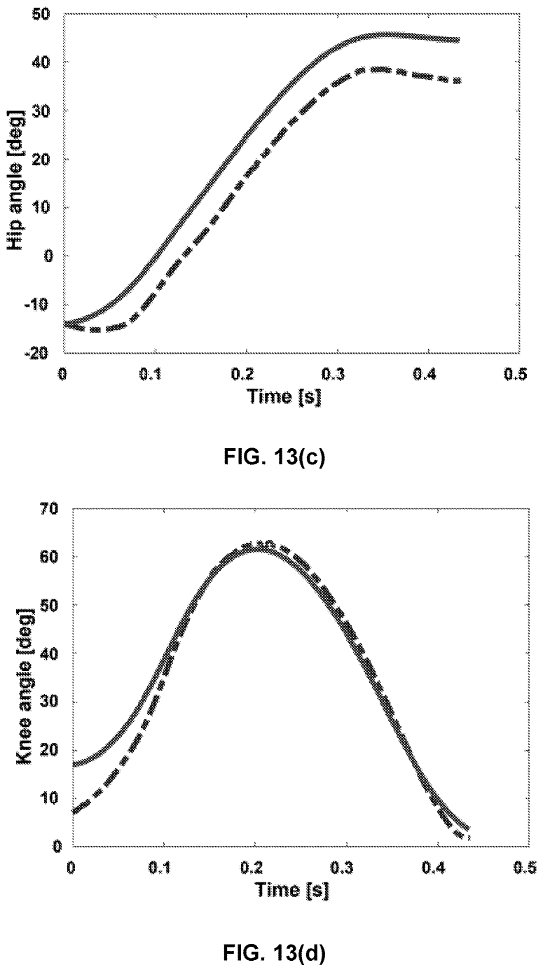

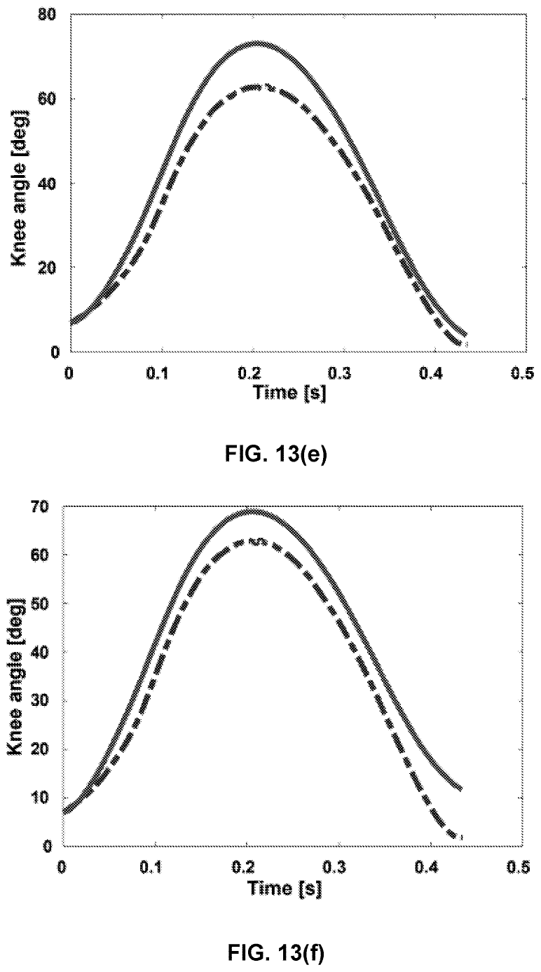

FIG. 13 shows the simulation results of the trajectories generated with different conditions. The kernel weights were used to calculate the hip and knee joint reference trajectories of the swing leg with different trajectory parameters in simulation. FIG. 13(a) and FIG. 13(d) simulated the case where the initial joint angle in the beginning of the swing phase had a relatively large deviation from the first point of the baseline trajectory. In this situation, if the baseline trajectory is directly copied to the reference, the gap between the initial point and the reference trajectory may cause an obvious jerk and lead to unstable exoskeleton control. The DMP provided a smooth transition for the exoskeleton control, where the reference trajectory converged to the baseline trajectory from the starting joint angle. In the case of r setting (see FIG. 13(b) and FIG. 13(e)), there was more obvious change in the peak angle (range of motion) of knee joint rather than in hip joint. This characteristic may be applied to the adjustment of step height. The scaling parameter for both hip and knee joint was set to default 1.0 in the following experiments because adjusting step height was not the focus of this study.

In FIG. 13(c) and FIG. 13(f), the respective ending positions of the hip and knee joints was changed by the goal offset g_{offset}. The DMP module was still able to allow the reference trajectory to successfully converge to the updated ending position while keeping the shape of reference trajectory consistent with the baseline trajectory. In the experiment, the goal offset of hip joint was used to adjust step length, and one of the knee joint was set to 0°.

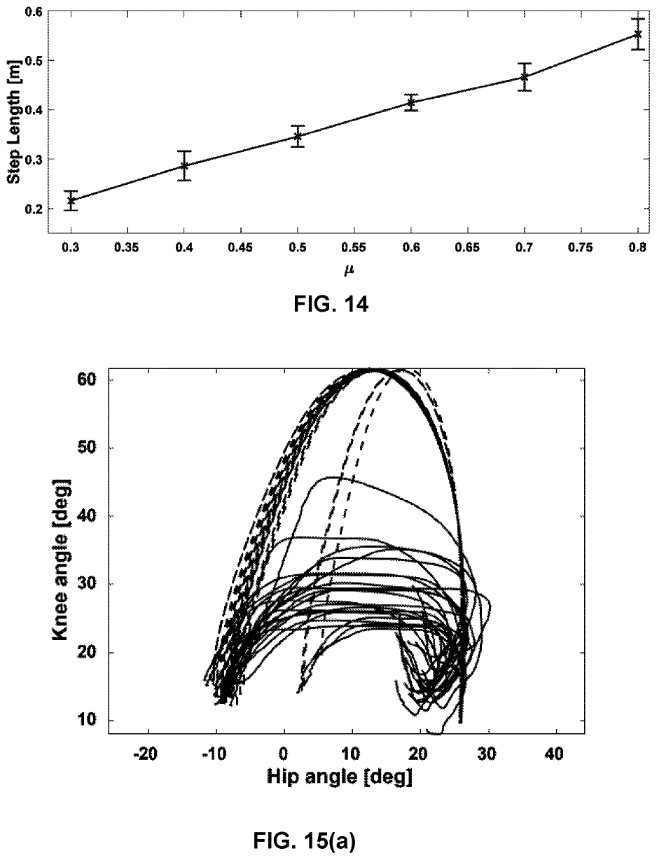

Feasibility of using DMP to scale the step length was studied. One subject was asked to walk on level ground with the exoskeleton assistance in different step length. Considering the fact that the experiment did not focus on the continuous adjustment of step length, several discrete values of the scaling variable μ were selected, and measured the actual step length in each step. It is noted that a continuous adjustment of step length as disclosed in earlier section may also be implemented according to other examples. The mean and standard deviation of step length over different scaling variable was presented in FIG. 14. The results show that the exoskeleton control with scaling the ending hip angle may indeed achieve the purpose of adjusting the step length.

In summary, the implementation of DMP in combination with the several parameters enables the generated reference trajectory to adapt to the different initial joint configuration and provide a flexible gait pattern adjustment, while keeping the reference trajectory as similar as possible to the baseline trajectory. In the following walking experiments, since the purpose was to validate the exoskeleton control part, the scaling variable μ was fixed at 0.7, and the scaling coefficient r for hip and knee were both set to 1.0.

### Evaluation of Exoskeleton Assistance

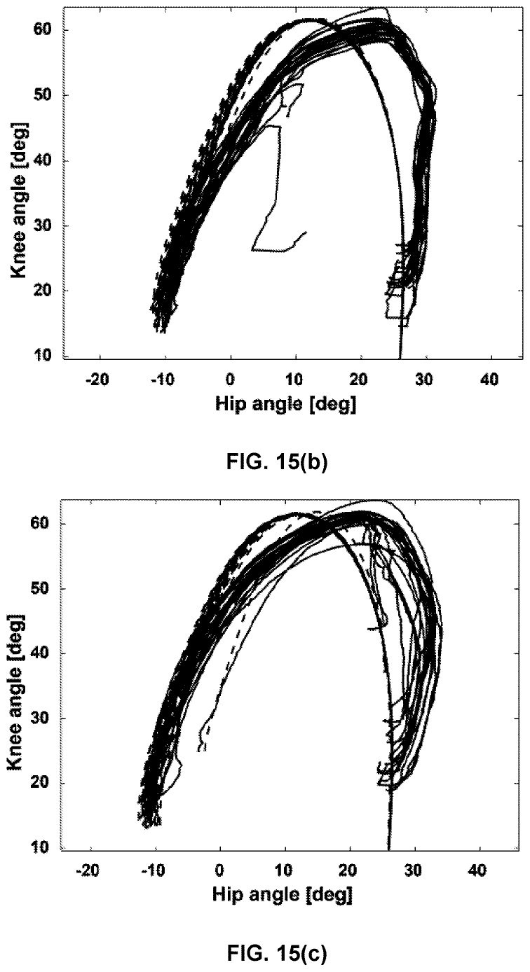

FIG. 15 shows the swing phase gait trajectory collected from one subject in different exoskeleton control conditions. The dashed line represents the reference trajectory generated by the DMP module, and the solid line represents the actual gait trajectory. There is slight difference among the reference trajectories, because the DMP module will adjust gait trajectory to match different initial joint position in the starting of the swing phase. In unassisted trials, the subjects' movement has poor consistency and relatively large deviations from the reference trajectories as the exoskeleton did not provide movement assistance. In the conditions of 2D and 4D path-based control, the assistance provided by the exoskeleton can guide the leg movement toward the desired swing phase path, and make the shape of gait trajectory more consistency.

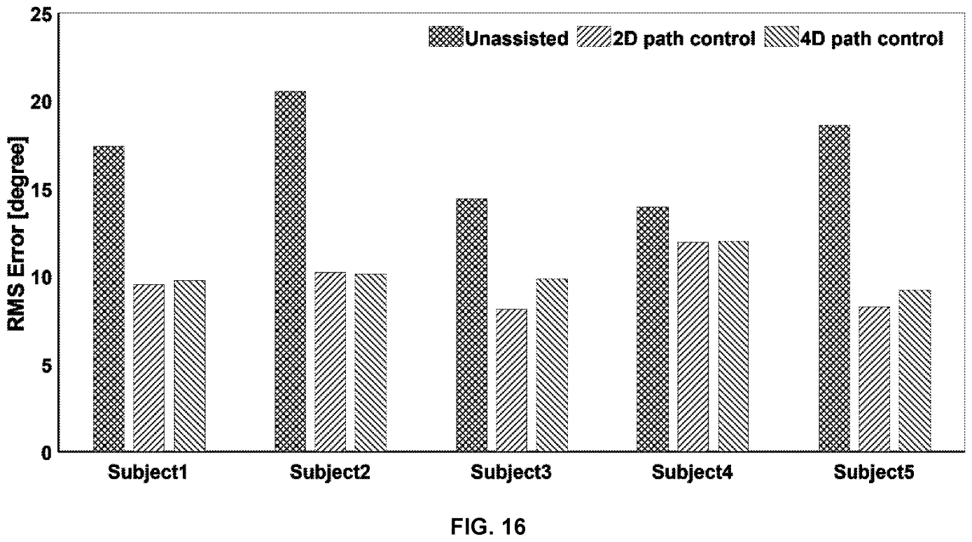

To quantitatively evaluate the effectiveness of exoskeleton assistive control, the trajectory root mean square (RMS) errors between the reference and actual movement were calculated, where the swing phase trajectory was normalized, interpolated and averaged over the duration of swing phase by considering the comparability. The mean trajectory RMS errors for all subjects and all control conditions are presented in FIG. 16. Among all subjects, the RMS error has a significant decline in both of path-based control trials. The mean RMS error for all subject was reduced from 16.97° in unassisted trials to 9.61° in 2D path control with p<0.05, and to 10.17° in 4D path control with p<0.05. It should be noted that there are no significant differences between 2D and 4D path control conditions. In addition, the altering of step length also indicates the improvement of the walking performance with the exoskeleton assistive control. The step length was relatively limited in the unassisted trials as the subjects' legs were hindered by the passive torque from the exoskeleton. The movement assistance provided by path control made the mean step length (shown in Table 1) increase from 0.380m to 0.482m (2D path) and 0.460m (4D path).

TABLE 1
The mean step length for all subjects in
different control conditions (unit: m)

| | unassisted | 2D control | 4D control |
|---|---|---|---|
| | (mean ± std) | (mean ± std) | (mean ± std) |
| S1 | 0.380 ± 0.0173 | 0.542 ± 0.0287 | 0.473 ± 0.0215 |
| S2 | 0.308 ± 0.0175 | 0.464 ± 0.0290 | 0.477 ± 0.0635 |
| S3 | 0.425 ± 0.0329 | 0.490 ± 0.0190 | 0.446 ± 0.0471 |
| S4 | 0.406 ± 0.0340 | 0.538 ± 0.0487 | 0.542 ± 0.0794 |
| S5 | 0.370 ± 0.0416 | 0.378 ± 0.0910 | 0.364 ± 0.0568 |
| all | 0.380 ± 0.0299 | 0.482 ± 0.0432 | 0.460 ± 0.0536 |

TABLE 2
The mean step time for all subjects in
different control conditions (unit: s)

| | unassisted | 2D control | 4D control |
|---|---|---|---|
| | (mean ± std) | (mean ± std) | (mean ± std) |
| S1 | 1.06 ± 0.171 | 1.39 ± 0.136 | 1.69 ± 0.286 |
| S2 | 0.97 ± 0.121 | 1.44 ± 0.316 | 1.32 ± 0.205 |
| S3 | 0.76 ± 0.046 | 1.10 ± 0.252 | 1.10 ± 0.371 |
| S4 | 1.45 ± 0.186 | 1.68 ± 0.203 | 1.62 ± 0.273 |
| S5 | 0.84 ± 0.104 | 1.03 ± 0.218 | 0.98 ± 0.178 |
| all | 1.02 ± 0.126 | 1.33 ± 0.225 | 1.34 ± 0.263 |

### Variance in Step Length and Time

As mentioned in previous sections, the aim of the proposed exoskeleton control is to provide movement assistance and guidance to reduce trajectory errors while still allowing subjects' effort to adjust gait pattern (step length and time). To evaluate the capability, the step length and time were measured in the three control conditions, and their variance was used to indicate the freedom which subjects had to control leg movement during walking. The mean and standard deviation of the step length and time for individuals and all subject in different control conditions are respectively shown in Table 1 and Table 2. Compared with the unassisted condition, nearly all subjects (except S4) showed significant increases in the step length in 2D and 4D path control conditions, and the mean step length over all subjects was improved from 0.380m to 0.482m (2D path) and 0.460m (4D path). The standard deviations of the step length in the path control conditions were not obviously declined, compared with the unassisted control condition. It indicates that subjects under the exoskeleton assistance can also adjust gait step length as freely as the condition without assistance. The mean step time has a certain increment in 2D and 4D path control trials. It may be due to the change in individual preferences caused by the intervention of exoskeleton control. In order to reduce the influence brought by the difference in the mean step time, the step time variability using the ratio of standard deviation to mean was assessed. The average ratio over all subjects in 2D path and 4D path control trials are 17.61% and 20.26%, which are not smaller than the value of 11.99% in the unassisted trials. According to the variance in the step length and time, it can be validated that the proposed assistive control does not limit subjects' control freedom for leg movement, but allows them to change the gait pattern by their own efforts.

### Stance and Swing Legs Synchronization

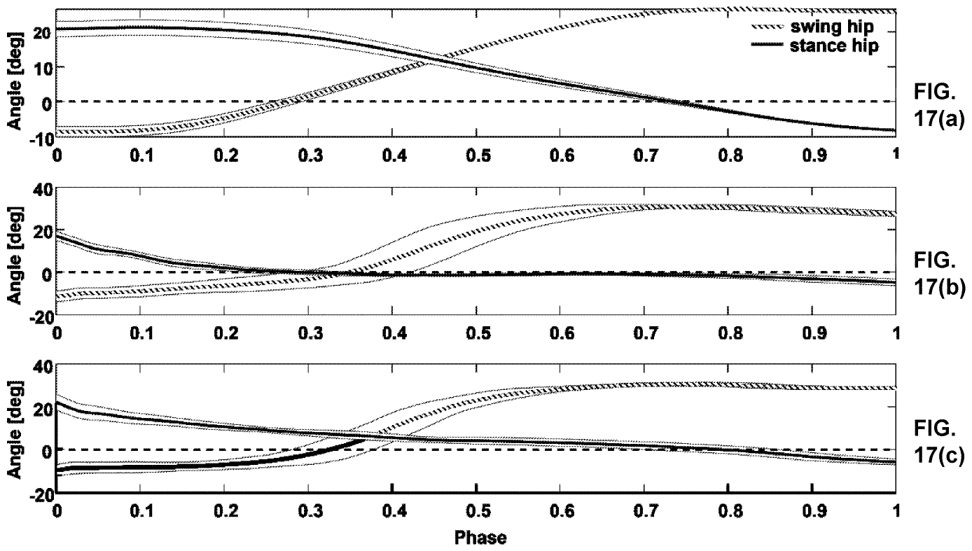

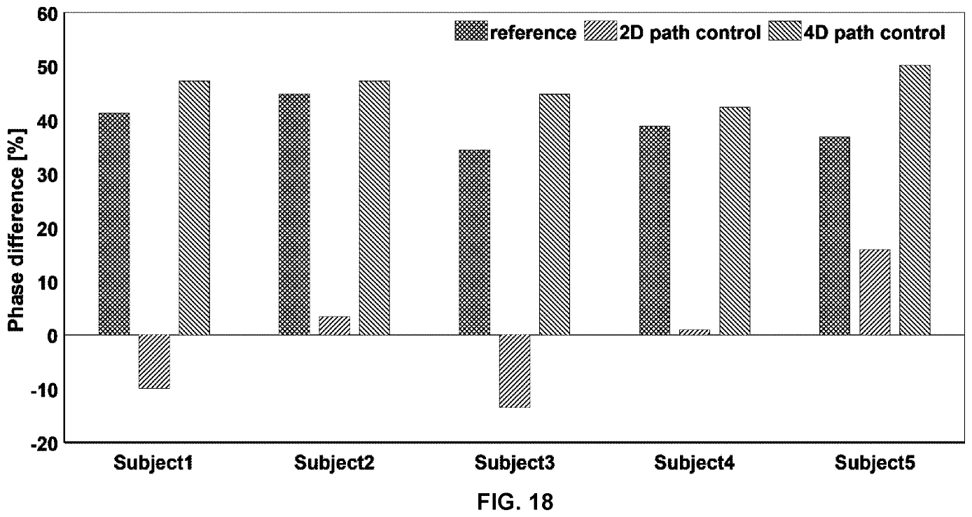

In this study, the aim of 4D path-based control is to synchronize the movement of swing and stance legs. Different from 2D path-based control it searches the target point in the 4D space consisting of hip and knee angles on two sides of the lower limb. The phase of crossing-zero point of hip joint was used to characterize the synchronization between swing and stance legs. FIG. 17 presents the reference and actual hip trajectories on swing and stance legs in the 2D and 4D path-based control conditions. Hip joint trajectories of swing and stance legs were normalized, interpolated and averaged over the swing phase duration. From the reference trajectories, it may be seen that the swing hip crosses over zero point ahead of the stance hip, and there is a certain phase difference. However, the phases of crossing-zero points of swing and stance hip joints in the case of 2D path based control are very closed because the swing and stance legs of the exoskeleton are controlled independently. To quantitatively evaluate the synchronization between swing and stance legs, the phase difference between the crossing-zero point of both legs was measured and shown in FIG. 18. The mean phase difference for all subjects in 4D path control is 46.4%, and closed to the value of 39.2% in the reference trajectory. However, the value in 2D path-based control is-0.6%, which means the stance hip even crossed over zero point earlier than the swing hip in some steps. Therefore, compared with the conventional 2D path control, the trajectory control with searching target point in 4D space is actually able to facilitate the synchronization of legs movement as its target trajectory depends on the movement of the swing and stance legs together.

All examples described herein, whether of apparatus, methods, materials, or products, are presented for the purpose of illustration and to aid understanding, and are not intended to be limiting or exhaustive. Modifications may be made by one of ordinary skill in the art without departing from the scope of the invention as claimed.

1. A gait trajectory system, comprising:
a controller configured to:
for a step of a user:
using a dynamic movement primitives-based trajectory generation module, determine a reference trajectory based on a set of initial joint angles, a goal offset, and a forcing term;
update a vector table based on the reference trajectory, wherein the vector table includes multiple sets of joint angles corresponding to at least one leg of the user;
determine from the vector table a set of closest joint angles closest to a set of current joint angles; and
determine from the vector table a set of target joint angles based on the set of current joint angles and a predetermined angular distance.

2. The system as recited in claim 1, wherein the controller is further configured to: iteratively: determine from the vector table the set of closest joint angles closest to the set of current joint angles; and determine from the vector table a set of target joint angles based on the set of current joint angles and a predetermined angular distance.

3. The system as recited in claim 1, wherein the controller is further configured to: beginning from a first index of the vector table towards an end of the vector table, determine from the vector table the set of closest joint angles closest to the set of current joint angles.

4. The system as recited in claim 3, wherein the controller is further configured to: update the first index to an index of the set of closest joint angles for a next iteration.

5. The system as recited in claim 1, wherein the controller is further configured to: beginning from a second index of the vector table towards an end of the vector table, determine from the vector table the set of target joint angles based on the set of current joint angles and the predetermined angular distance, wherein the second index is an index of the set of closest joint angles in the vector table.

6. The system as recited in claim 1, wherein the controller is further configured to: adjust the set of current joint angles towards the set of target joint angles.

7. The system as recited in claim 1, wherein the controller is further configured to: determine an end point of the step responsive to the current joint angles being within a threshold from a last set of joint angles of the vector table; and proceeding to a next step.

8. The system as recited in claim 1, wherein the vector table comprises multiple sets of joint angles corresponding to both legs of the user.

9. The system as recited in claim 1, wherein the multiple sets of joint angles comprise multiple hip angles and multiple knee angles.

10. The system as recited in claim 1, wherein the goal offset corresponds to a difference between an ending point of the reference trajectory and a baseline trajectory.

11. The system as recited in claim 10, wherein the goal offset corresponds to a difference between a hip joint angle of the user and a hip joint angle from a database.

12. The system as recited in claim 1, wherein the controller is further configured to: determine the goal offset based on a step length of the user.

13. The system as recited in claim 1, wherein the forcing term comprises kernel weights obtained from a pre-trained machine learning model.

14. The system as recited in claim 1, wherein the forcing term comprises a scaling coefficient corresponding to an ending hip angle of a previous step.

15. The system as recited in claim 14, wherein the controller is further configured to: determine the scaling coefficient based on an ending hip angle of a previous step of the user.

16. The system as recited in claim 1, further comprising a wearable lower limb exoskeleton having at least one joint actuator attached to a swing leg of the user, wherein the controller is further configured to: drive the at least one joint actuator based on the set of target joint angles.

17. The system as recited in claim 16, wherein the controller is further configured to: leave a stance leg of the user unactuated.

18. The system as recited in claim 16, further comprising: a plurality of joint angle sensors for determining a hip joint angle and a knee joint angle of the at least one leg of the user.

19. The system as recited in claim 16, wherein the lower limb exoskeleton includes at least one hip joint actuator and at least one knee joint actuator.

20. A gait trajectory control method, comprising:
for a step of a user:
using a dynamic movement primitives-based trajectory generation module, determining a reference trajectory based on a set of initial joint angles, a goal offset, and a forcing term;
updating a vector table based on the reference trajectory, wherein the vector table includes multiple sets of joint angles corresponding to at least one leg of the user;
determining from the vector table a set of closest joint angles closest to a set of current joint angles; and
determining from the vector table a set of target joint angles based on the set of current joint angles and a predetermined angular distance.
